(19) United States
(12) Patent Application Publication
(10) Pub. No.: US 2004/0007232 A1
(43) Pub. Date: Jan. 15, 2004

(54) Method of regulating the open-loop pressure of a repiratory assistance apparatus

(76) Inventor: Jean-Denis Rochat, Genolier (CH)

Correspondence Address: STURM & FIX LLP, 206 SIXTH AVENUE, SUITE 1213, DES MOINES, IA, 50309-4076, US

(21) Appl. No.: 10/464,911

(22) Filed: Jun. 19, 2003

Related U.S. Application Data
(63) Continuation of application No. PCT/IB01/02628, filed on Dec. 20, 2001.

(30) Foreign Application Priority Data
Dec. 22, 2001 (EP) 00811238.5

(51) Int. Cl. A61M 16/00; A62B 7/00

(52) U.S. Cl. 128/205.24; 128/204.18

(57) ABSTRACT

The invention concerns a method which consists in connecting a floating element adjusting the passage section of said valve to the body of said valve by elastic guide means and to a field coil of said electrodynamic control, exerting on said floating, adjusting element a reaction force tending to balance the pressure exerted on said floating adjusting element by the gas of said source and powering said field coil continuously calculating the instantaneous intensity and the supply current direction on the basis of the differential pressure between the supply pressure and the set pressure of compressed air of said instantaneous flow rate and of the constants of said apparatus.

The present invention relates to an open-loop pressure regulation method for a respiratory assistance apparatus supplied by a pressurized respiratory gas source fitted with a regulating valve with electrodynamic control, and to a respiratory assistance apparatus for implementing this method.

The problem encountered with respiratory assistance apparatuses which are required to supply a variable air flow rate at constant pressure is that of the response time. It is fact necessary to succeed in producing an endotracheal reference pressure which can be adjusted by the practitioner, which is independent of the instantaneous inhalation flow rate demanded by the patient, the exhalation passing through an exhalation valve while the inhalation valve is closed.

There are two types of respiratory assistance apparatus. The apparatuses of the first type comprise a supply of pressurized respiratory gas, the flow rate and the pressure of which are regulated by a regulating valve with a variable constriction. The apparatuses of the second type have no pressurized gas supply, but a variable pressure and flow rate compressor.

Existing apparatuses operate with pressure feedback, which requires a compromise between stability of the system in closed-loop mode and its response time. The response time of such systems is about 50 to 150 ms, while the response time of the valve is about 4 to 10 ms.

In order to be able to operate in open-loop mode, it is necessary first of all to find a regulation system operating without mechanical friction, given that this is a virtually uncontrollable variable, such that it is then essential in such a case to have “feedback” to avoid uncontrollable changes in the air supply.

The aim of the present invention is to make it possible to regulate the pressure of a respiratory assistance apparatus in open-loop mode.

To this end, the subject of this invention is a method of regulating pressure in open-loop mode in a respiratory assistance apparatus supplied by a source of pressurized respiratory gas fitted with an electrodynamic regulating valve with a variable constriction, as claimed in claim 1. The subject of the invention is also a respiratory assistance apparatus as claimed in claim 2.

The advantage of this method and of the respiratory assistance apparatus resides in the fact that it is enough to measure the supply pressure and the instantaneous flow rate and to know the reference pressure in order to supply the driving coil of the solenoid valve with the instantaneous current corresponding to the instantaneous constriction which is a function of the instantaneous flow rate demanded.

The appended drawing shows, schematically and by way of example, one embodiment of a respiratory assistance apparatus and of the regulation system for this apparatus, for implementing the regulation method which is the subject of the present invention.

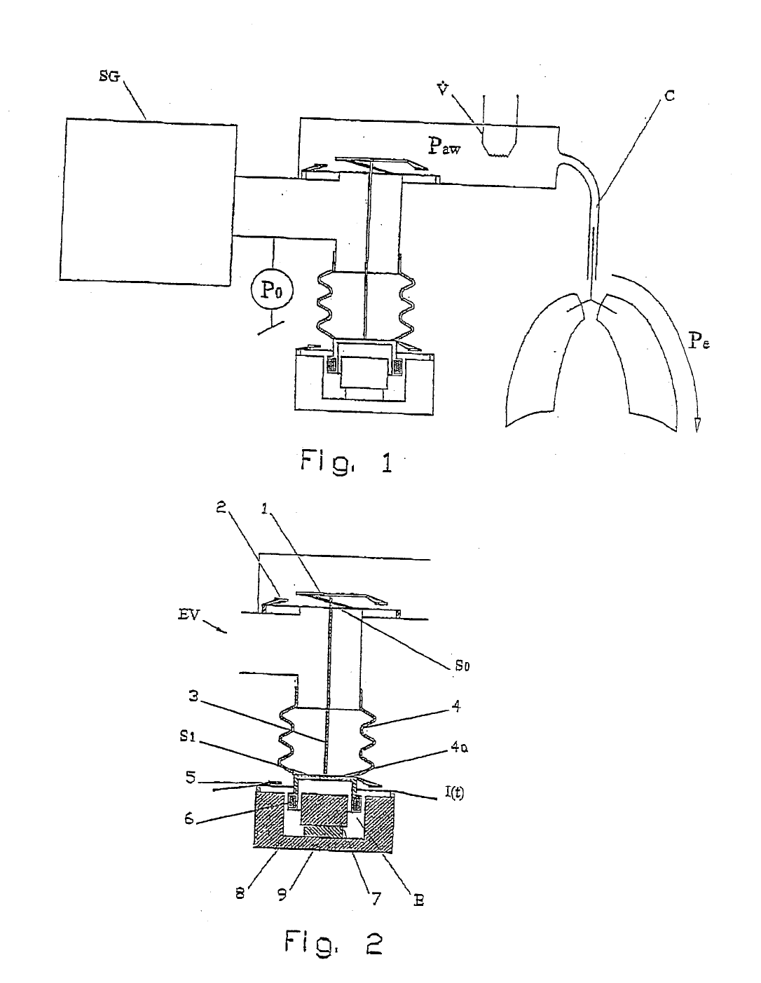

FIG. 1 is a diagram of this embodiment;

FIG. 2 is an enlarged partial view of FIG. 1, relating to a regulating valve.

The respiratory assistance apparatus illustrated comprises a pressurized respiratory gas source SG, a solenoid valve EV for regulating the cross section for passage of the pressurized gas and a cannula C intended to be inserted into the patient's trachea. A sensor measures the pressure P₀ upstream of the solenoid valve EV and another sensor measures the flow rate \dot{V}.

To be able to achieve regulation in closed-loop mode, it is necessary to virtually eliminate mechanical friction, given that this is not constant and so prevents such regulation.

It is for this reason that it is necessary to make sure that the solenoid valve operates virtually without mechanical friction. To this end, the solenoid valve illustrated in FIG. 2 comprises a seat of cross section S₀ closed by a flap 1. This flap 1 is suspended by a spring guide 2 with three or more arms fastened to the periphery of the valve seat S₀. This flap 1 is connected by piano wire 3 to the bottom 4a of a bellows 4 of cross section S₁ which is substantially identical to cross section S₀ of the valve seat. Given that the flap 1 and the bottom 4a of the bellows 4 have substantially the same cross section and are subject to the pressure P₀ of the supply source, virtual equilibrium is established between the action of this pressure P₀ on the flap 1 and the reaction exerted on the bottom 4a of the bellows, such that the resultant is

P₀(S₀−S₁)≅0

The bellows 4 is very flexible in order to interfere as little as possible with the moveable system of the solenoid valve. The bottom 4a of the bellows 4 is suspended by a spring guide 5 identical to the spring guide 2. The bottom 4a of the bellows 4 bears a cylindrical coil 6 placed in a gap E made between a soft iron core 7 and a soft iron yoke 8 which are connected to the respective poles of a permanent magnet 9. This device for actuating the solenoid valve consists of an electrodynamic motor where the magnetic force is essentially independent of the coil position.

The cylindrical coil 6 is connected to a supply of current I, the instantaneous value I(t) of which is determined as a function of the reference pressure and of the instantaneous flow rate demanded by the patient.

Newton's law applied to the flap of the solenoid valve is:

ΣF=m·a=mÿ(t)

P(S₀−S₁)−P_{aw}S₀−F_{magn}+ky(t)+η\dot{y}(t)=mÿ(t)

where

η=mechanical strength of the bellows and of the spring guides

k=spring constant of the system

P_{aw}=RV(t)+R₂V²(t)+Pₑ

where: R, R₂ represent the resistances of the cannula to the flow of pressurized gas

Pₑ is the endotracheal reference pressure

The magnetic force on the flap is:

F_{magn}=B·l·I(T) irrespective of y(t)

giving the control current:

$$I ( t ) = \frac{1}{B} ( {P}_{0} \cdot ( {S}_{0} - {S}_{1} ) - {P}_{aw} {S}_{0} + k \cdot y ( t ) + η \dot{y} ( t ) - m \ddot{y} ( t ) )$$

The pressure P₀ is measured by the supply pressure sensor, while the pressure P_{aw} is only measured for reasons of safety, but its measurement would not be necessary within the scope of the method according to the invention. Given that P₀S₀≅0

$$I ( t ) = \frac{1}{Bl} [ - {S}_{0} ( R \cdot \dot{V} ( t ) + {R}_{2} \cdot {V}^{2} ( t ) + {P}_{c} ) + Icy ( t ) + η \dot{y} ( t ) - m \ddot{y} ( t ) ]$$

\dot{V} is a flow rate which can be measured for example with a hot wire anemometer. It would also be possible to measure this flow rate according to y(t) and ΔP, thus saving a flow rate sensor, but the specified range of operation would thereby be limited.

It can therefore be seen that the instantaneous opening of the solenoid valve according to the invention can be regulated in open-loop mode, given that the variables involved in the calculation of the instantaneous current I(t) are measurable variables, the other parameters being constants of the respiratory assistance device. Thus the response time is that of the flap 1 of the solenoid valve, which is about 4 to 10 ms.

1. A method of regulating pressure in open-loop mode in a respiratory assistance apparatus supplied by a pressurized respiratory gas source (SG) fitted with a regulating valve (EV) with electrodynamic control, characterized in that a floating element (1) for regulating the passage cross section of said valve is connected on the one hand to the body of said valve by springy guiding means (2), and on the other hand to a driving coil (6) of said electrodynamic control, in that a reaction force tending to balance the pressure exerted by the gas from said source (SG) is exerted on this floating regulating element (1) and in that said driving coil (6) is supplied by continuously calculating the instantaneous value I(t) and the direction of the supply current as a function of the pressure difference between the supply pressure P₀ and the reference pressure Pₑ of the compressed air, of said instantaneous flow rate and of the constants of said apparatus.

2. The method as claimed in claim 1, characterized in that, in order to exert on said floating regulating element (1) said reaction force tending to balance the pressure exerted by said gas, a second opposing floating element (4a) is formed which is dimensioned so that the force resulting from the pressure of said gas exerted thereon is substantially equal to that exerted on said floating element (1), this second floating element (4a) is connected to a pipe for said gas by means of a sealed bellows (4) and said floating elements (1, 4a) are connected kinematically to each other.

3. A respiratory assistance apparatus supplied by a pressurized respiratory gas source fitted with an electrodynamically driven valve for regulating the flow rate of said gas combined with control means in open-loop mode, characterized in that said valve (EV) comprises a moveable element comprising an element for regulating the flow rate of said gas (1) which is subjected to the pressure of this gas and is connected. to the body of said valve by springy guiding means (2), means (3, 4, 4a) for transmitting to said regulating element (1) a reaction force at the most equal to that exerted on said regulating element (1) by said pressurized gas and a driving coil (6) engaged in a gap (E) oriented coaxially to the direction of displacement of said moveable element and connected to a supply source (I(t)) combined with said control means.

4. The apparatus as claimed in claim 3, characterized in that said means (3, 4, 4a) for transmitting to said regulating element (1) said reaction force comprise a bellows (4), one end of which communicates in a sealed manner with said pressurized gas supply source (SG) and the other end of which has a bottom (4a) separating said pressurized gas from the atmosphere, the bottom (4a) of said bellows (4) being placed opposite said element (1) for regulating the flow rate, said bottom (4a) and said regulating element (1) being connected to each other by a connection element (3) and in that said driving coil (6) is secured to the outer part of said bellows (4).

5. The apparatus as claimed in either of claims 3 and 4, characterized in that said driving coil (6) and said gap (E) form an electrodynamic motor.
